SEQUENCE LISTING (9 sequences)

SEQ ID NO: 1 - DNA, 15 bp, synthetic construct
  source 1..15: mol_type = other DNA
ctgggcaattactcg

SEQ ID NO: 2 - DNA, 15 bp, synthetic construct
  source 1..15: mol_type = other DNA
ctcattgtaactcct

SEQ ID NO: 3 - DNA, 15 bp, synthetic construct
  source 1..15: mol_type = other DNA
aagttcactctttgc

SEQ ID NO: 4 - DNA, 15 bp, synthetic construct
  source 1..15: mol_type = other DNA
tgttcccgctcaact

SEQ ID NO: 5 - DNA, 15 bp, synthetic construct
  source 1..15: mol_type = other DNA
tacgcctataacttg

SEQ ID NO: 6 - DNA, 15 bp, synthetic construct
  source 1..15: mol_type = other DNA
tcctttcctgatagg

SEQ ID NO: 7 - DNA, 15 bp, synthetic construct
  source 1..15: mol_type = other DNA
tctcagacctccgta

SEQ ID NO: 8 - DNA, 15 bp, synthetic construct
  source 1..15: mol_type = other DNA
gcgcaacttgatgat

SEQ ID NO: 9 - DNA, 15 bp, synthetic construct
  source 1..15: mol_type = other DNA
tcccttgcgatttac

(19) United States
(12) Patent Application Publication
(10) Pub. No.: US 2026/0326103 A1
(43) Pub. Date: Oct. 22, 2026

(54) SINGLE CELL COMBINATORIAL INDEXING

(71) Applicant: SCALE BIOSCIENCES INC., San Diego, CA (US)

(72) Inventors: Frank STEEMERS, Encinitas, CA (US); Fan ZHANG, Menlo Park, CA (US); Kevin GUNDERSON, Encinitas, CA (US)

(21) Appl. No.: 19/119,390

(22) PCT Filed: Dec. 22, 2023
(86) PCT No.: PCT/US2023/085820

§ 371 Date: Apr. 8, 2025

Related U.S. Application Data
(60) Provisional application No. 63/435,788, filed on Dec. 28, 2022.

(51) Int. Cl. B01L 3/00; C12Q 1/6869

(52) U.S. Cl. CPC B01L 3/502761; C12Q 1/6869; B01L 2200/0668; B01L 2300/0829; B01L 2300/0858; B01L 2300/161

(57) ABSTRACT

This disclosure provides, among other things, an assembly comprising a macrowell plate that comprises a single open-bottomed well and a microwell plate. In the assembly, the bottom of the macrowell plate is affixed to the top of the microwell plate and multiple microwells of the microwell plate are exposed at the bottom of the well of the microplate, and in some embodiments, the wells of the microwell plate are dimensioned to allow a single bead of a defined diameter into a well but comprise a constriction that prevents the bead from coming into contact with the bottom of the well. In an alternate configuration, the wells of the microplate may be pre-barcoded by releasing oligonucleotides from barcoded beads in the wells. Kits and methods are also provided.

### CROSS-REFERENCING

This application claims the benefit of U.S. provisional application Ser. No. 63/435,788, filed on Dec. 28, 2022, which application is incorporated by reference for all purposes.

### INCORPORATION BY REFERENCE OF SEQUENCE LISTING PROVIDED AS A SEQUENCE LISTING XML FILE

A Sequence Listing is provided herewith as a Sequence Listing XML, SCBI-009WO_SEQLIST, created on Dec. 22, 2023, and having a size of 8,864 bytes. The contents of the Sequence Listing XML are incorporated herein by reference in their entirety.

### BACKGROUND

Cell-specific barcodes can be added to cells using a “split and pool” approach. Such methods may involve partitioning cells into compartments (such that each compartment receives multiple cells), adding barcode subunits to the cells while they are compartmentalized (where each compartment may receive a different subunit), pooling the cells, and then repeating the process as many times as desired. If the partitioning is random, then each cell should receive a different set of subunits by the end of the process. Because the total number of indexing combinations grows exponentially with the number of the split and pool cycles, the split and pool approach can be used to index an almost unlimited number of cells. Examples of split and pool methods for labeling proteins and RNAs on a cell-by-cell basis are described in O'Huallachain et al (Commun. Biol. 2020 3:279) and Cao et al (Science. 2017 357:661-667), among many others.

One problem with the way in which conventional split and pool methods are typically performed is that the pooling and partitioning steps require a significant number of liquid transfer steps. For example, pooling and re-partitioning a 96-well plate to a single tube and back again requires 96 individual transfers into the tube, then another 96 transfers back. In theory, a conventional liquid handling robot and/or a multichannel pipette could be used. However, the non-parallel nature of split and pool approach makes such conventional robots and/or pipettes less than ideal. Additionally, liquid transfer steps can lead to loss of sample, which can cause problems in some cases.

This disclosure provides a solution to this problem.

### SUMMARY

This disclosure provides, among other things, an assembly comprising: (a) a macrowell plate that comprises a single open-bottomed well and (b) a microwell plate comprising wells that have entrances that are in the range of 10-200 microns in diameter. In the assembly, the bottom of the macrowell plate is affixed to the top of the microwell plate such that at least 1,000 microwells of the microwell plate are exposed at the bottom of the well of the microplate. In some embodiments, the wells of the microwell plate are dimensioned to allow a single bead of a defined diameter into a well but comprise a constriction that prevents the bead from coming into contact with the bottom of the well. In other embodiments, the wells of the microwell plate are dimensioned to allow only a single bead of a defined diameter to load into the well, after which the barcode oligos on the loaded bead are transferred to the surface of the well.

Kits and methods of use are also provided.

### BRIEF DESCRIPTION OF THE FIGURES

Some aspects of the technology described herein may be best understood from the following detailed description when read in conjunction with the accompanying drawings. It is emphasized that, according to common practice, the various features of the drawings are not to scale. Indeed, the dimensions of the various features are arbitrarily expanded or reduced for clarity. Included in the drawings are the following figures.

FIG. 1 illustrates an embodiment of the present assembly.

FIGS. 2A and 2B illustrate exemplary microwells geometries that allow a single bead of a defined diameter into a well but comprise a constriction that prevents the bead from coming into contact with the bottom of the well.

FIGS. 3A-3E illustrate various configurations of a macrowell plate. FIG. 3A shows the footprint and arrangement of wells in a standard microtiter plate. FIGS. 3B-3E illustrate examples of macrowell plates that can be part of the present assembly. As shown, the size of the open bottomed well (or “macrowell”; MaW) of the macrowell plate can be selected depending on how the assembly is going to be used. The single open-bottomed well can be any selected size, in any position, and any shape. In the illustrated macroplates, the MaW region is a passage that extends from the top of the plate to the bottom of the plate; i.e., it forms a passageway and is “open”. The hatched area is not open.

FIG. 4 illustrates how the interior walls of the wells of a microwell plate (the “microwells”) may be coated with a hydrophilic material and the top surface of the microwell plate may be coated in a hydrophobic material. These coatings facilitate distribution of a liquid that has been placed on top of the microwell plate into the microwells.

FIG. 5 illustrates an exemplary workflow in which the present assembly may be used.

FIG. 6 illustrates an exemplary molecular biology workflow for the analysis of proteins.

FIG. 7 illustrates an exemplary molecular biology workflow for analysis of mRNA.

FIGS. 8A and 8B illustrate the generation of barcode microwell arrays and their use a two-level combinatorial indexed assays. FIG. 8A: Generation of pre-barcoded microwell arrays. FIG. 8B: An example of an implementation of two-level split/pool and combinatorial barcoding (SCI-Seq, QBC, etc.) on pre-barcoded microwell arrays.

FIG. 9 illustrates an exemplary molecular biology workflow for the analysis of proteins. The antibody oligonucleotide conjugate can be directly captured on a barcoded bead without the need for a splint or accessory oligonucleotide.

FIG. 10 illustrates a macrowell plate that provides access to the microwell plate for column 1.

FIGS. 11A and 11B show exemplary data obtained from a single cell protein profiling assay experiment produced using the present method.

FIGS. 12A and 12B show exemplary data from: (FIG. 12A) cell hashing experiments demonstrating single cell resolution detection of proteins and (FIG. 12 B) CD4 and CD8 single cell protein sequencing data transformed into a ‘flow’ plot. Each dot represents a single cell. Single cells either display CD4 or CD8 but are not expressed simultaneously in the same cell.

FIG. 13 shows a single cell sequencing library generation using a lysis buffer with and without additives

FIG. 14 Ab-oligonucleotide panel used in the studies. From top to bottom, SEQ ID NOS: 1-9.

FIG. 15 illustrates an example of a microwell array.

FIGS. 16A-16C illustrate microwell pages. FIG. 16A: single cells loaded into a microwell plate. FIG. 16B. 30 μm magnetic beads loaded into a microwell plate containing cells. FIG. 16C: Magnetic beads collected from the microwell plate (beads removed). Plate was pretreated using pluronic and BSA for 2 hrs, BSA 1 hour, cells stained by NucGreen.

FIG. 17 shows that beads can be used to capture cDNA or mRNA that has diffused out from microwells into the bulk solution above the microwells. Use of the beads increased the number of sequence reads from the cells by >10%. This method enables higher density microwell configurations while minimizing well-to-well cross talk.

FIGS. 18A and 18B show exemplary data obtained from a single cell whole transcriptome 3′ RNA profiling assay experiment produced using the present method. Briefly, the protocol starts by allocating fixed cells or nuclei to the wells of one or more 96-well plates. One sample per well or one sample distributed over multiple wells. The first index is introduced during reverse transcription with barcoded oligo-dT primers. Cells or nuclei are then pooled and loaded onto the microwell plate for a second level of indexing FIG. 18A: exemplary ‘barnyard’ plot for mouse and human cells. FIG. 18B: Barcode rank plot with reads per cell.

### DETAILED DESCRIPTION

Before embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a nucleic acid” includes a plurality of such nucleic acids and reference to “the compound” includes reference to one or more compounds and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

All patents and publications, including all sequences disclosed within such patents and publications, referred to herein are expressly incorporated by reference.

The headings provided herein are not limitations of the various aspects or embodiments of the invention.

### Assemblies

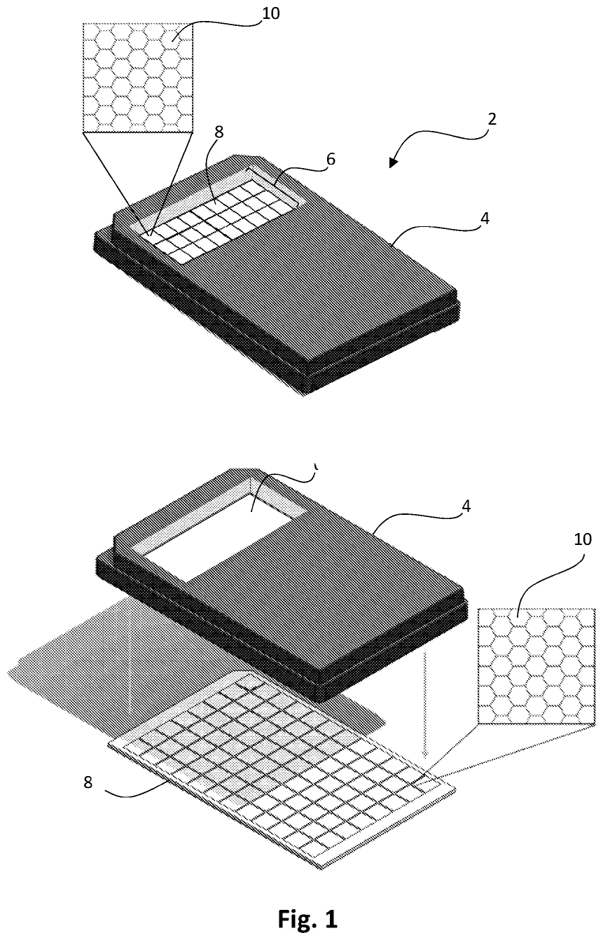

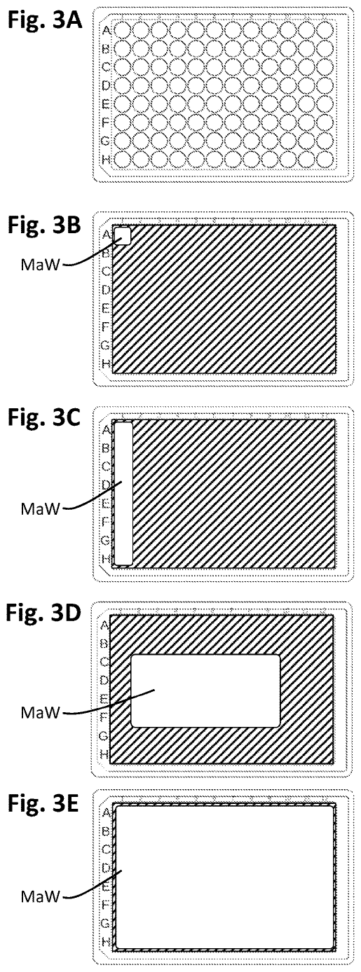

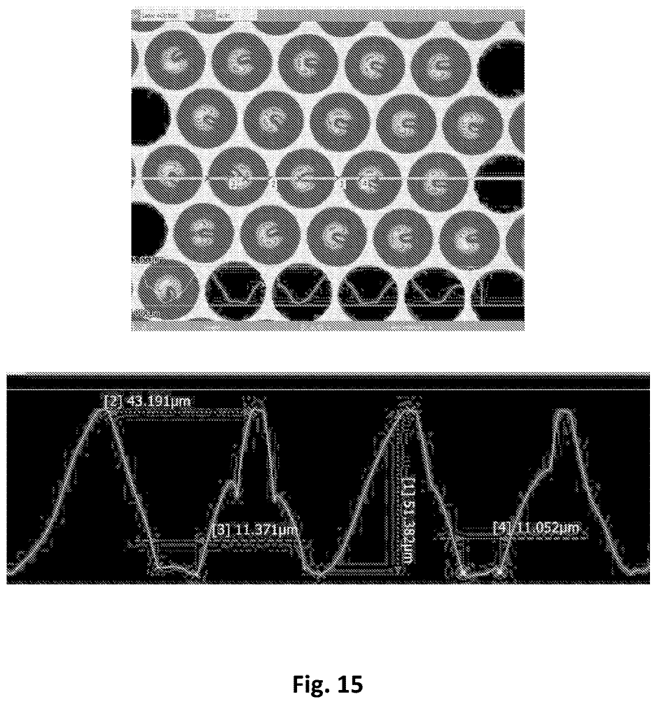

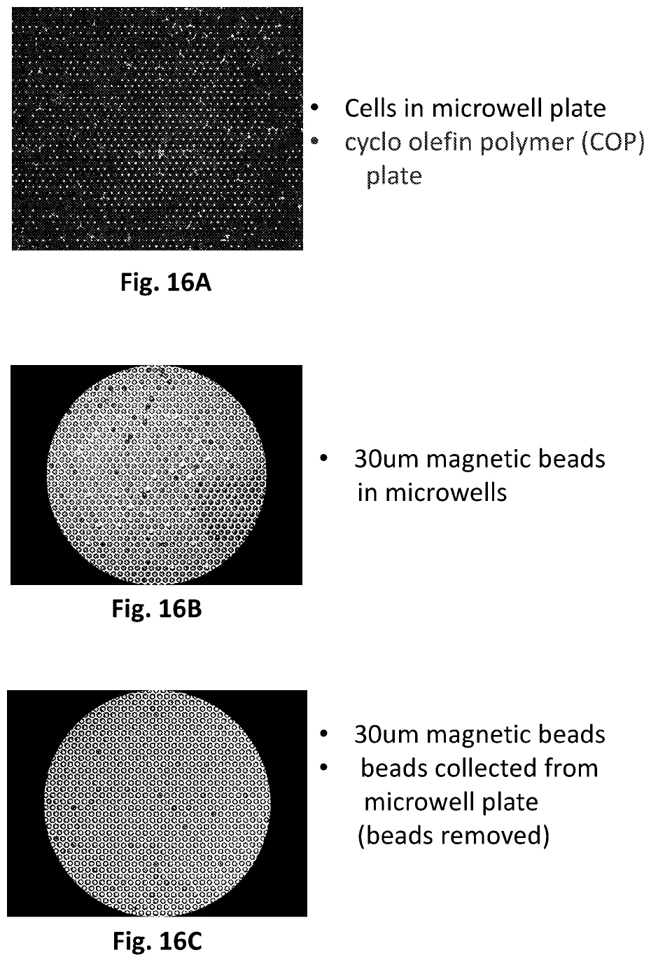

With reference to FIG. 1, assembly 2 may comprise: macrowell plate 4 and microwell plate 8. As illustrated, macrowell plate 4 comprises single open-bottomed well 6 (i.e., an opening that passes through the plate, referred to as the “macrowell” herein), which, as shown in FIG. 3, may vary in size, position, and shape. Wells 10 of microplate 8 (which may be referred to as “microwells” herein) have entrances that are in the range of 10-200 microns in diameter (e.g., 30-100 microns in diameter) and are of a volume in the range of 1-1,000 μL, e.g., 10-800 μL or 30-500 μL. In some embodiments, microwells 10 can be spaced apart by a suitable distance, e.g., a distance in the rage of 2-100 μm (e.g., in the range of 5-20 μm). In some embodiments, the spacing between the microwells may be as small as possible, thereby allowing the microwells to be as dense as possible (see, e.g., FIG. 15). In addition, the microwells may be arranged relative to one another in any suitable way, e.g., in a regular grid or a honeycomb (as illustrated), etc. The density of the microwells may be in the range of 200-1200 per mm² (e.g., 600-1000 per mm²). In some embodiments, the microwell plate may have a total of at least 500,000, at least 1M, at least 5M, or at least 10M microwells.

In the assembly, the bottom of the macrowell plate is affixed to the top of the microwell plate, e.g., bonded using an adhesive, or using clamps, laser or chemically bonded, clips, or another type of engaging element, etc. In some embodiments, the microwell plate has areas without microwells to facilitate bonding or improve bonding with the macrowell plate. In some embodiments, the width of the area without wells is large enough to facilitate accurate alignment and macrowell placement. In some embodiments, the microwell plate has markers (e.g. fiducial markers) to enable alignment with the macrowell plate. The process of bonding a macrowell plate to areas without microwells avoids partial or incomplete microwells. In some embodiments, parts of the macrowell plate overlaps with microwells. The number of microwells 10 that are exposed at the bottom of macrowell 6 may vary depending on the diameter of macrowell 6 and the density of the microwells 10. However, in general, at least 10,000, at least 50,000, at least 100,000, at least 500,000, at least 1M or at least 5M microwells 10 are exposed at the bottom of macrowell 6. In some embodiments, assembly 2 may additionally contain a gasket between macrowell plate 4 and microwell plate 8 that surrounds the bottom of open-bottom well 6, thereby preventing a liquid that has been contacted with the exposed microwells via the macrowell from coming into contact with unexposed microwells. In some embodiments, the microwell and microwell assembly are build as one piece and no assembly is required.

The present assembly may have dimensions that are the same as a standard microtiter plate, which has a length in the range of 127-128 mm (e.g., 127.71 mm), a width in the range of 85-86 mm (e.g., 85.43 mm) and, in some cases, a height in the range of 14.0-14.5 mm (e.g., 14.10 mm). As shown, the microplate and the macroplate may have a similar footprint, i.e., a length in the range of 127-128 mm and a width in the range of 85-86 mm. As such, in many embodiments, only a fraction of the total number of microwells of the microwell plate will be exposed in the macrowell. Specifically, in some embodiments, 1% to 95% (e.g., 1%-10%, 5%-20%, 20%-60%, 40%-80% or 70%-95%) of the total number of microwells in the microplate are exposed through the macrowell. Depending on how the assembly is fabricated, the microplate may have a height of 0.5 mm-2 mm, although assemblies that are taller or shorter may be used in certain circumstances. In some embodiments, the present assembly may have dimensions that are larger than a standard microtiter plate. By scaling the dimensions, a larger number of microwells can be accommodated. At least 1M, at least 5M, at least 10M, at least 20M, at least 50M or at least 100M microwells are exposed at the bottom of the macrowell.

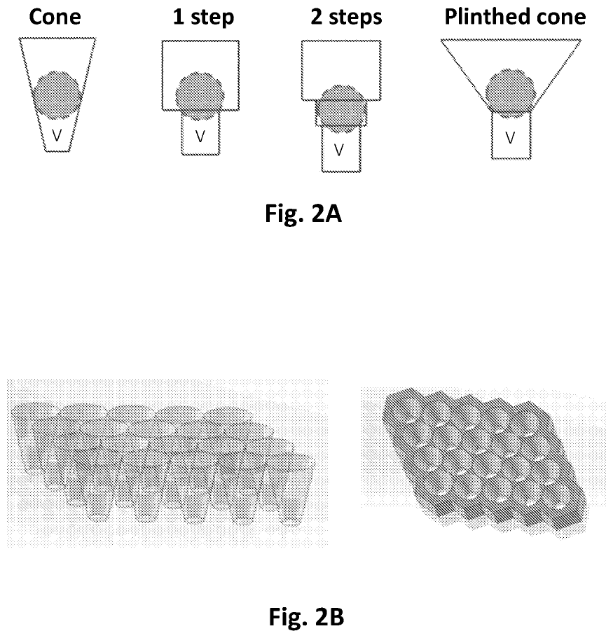

As noted above, the wells of the microwell plate are dimensioned to allow a single bead of a defined size into a well but, in some embodiments, the wells may comprise a constriction that prevents the bead from coming into contact with the bottom of the well (see, e.g., U.S. Pat. No. 8,257,967B2 and Walt, Science 2000 287:451-452, US20020160363A1). The microwells do not have walls that are continuously parallel from top to bottom. Rather, in some embodiments, the diameter of the microwells may be smaller at the bottom of the well than at the entrance of the well such that a bead that has a diameter of, e.g., 20%-80% of the diameter of the opening of a well will sink until it reaches a constriction, at which point it will stop moving. For example, in some embodiments the diameter at the bottom of the microwells (excluding any curved surface at the bottom) may be less than 10%, less than 20%, less than 30%, less than 50%, less than 70%, or less than 80%) of the diameter of the entrance to the well (or the diameter of the bead). As illustrated in FIG. 2A, this constriction may be accomplished by designing the wells as a cone (i.e., which continuously narrow towards the bottom), or to have a single step, two or more steps, a variation of a cone, or any combination thereof. Other designs would be apparent. Various well densities and well dimensions can be envisioned. Wells can be wide and shallow, or wells can be narrow and deep or any configuration in between to have a volume that can hold multiple cells. In some embodiments, the well can accommodate multiple cells or nuclei and simultaneously accommodate a bead, minimizing having wells with cells without a bead or wells with beads without cells. Cells in wells but without a bead will not convert into single cell sequencing libraries and results in loss and lower cell capture. In some embodiments, the microwell array and individual wells capture the majority of cells, and enable placement of the bead in the microwell, without the cells interfering in the bead placement process. Cell loading and distribution in the microwell array should follow a Poisson distribution. The well dimensions and volume should be designed accommodate a desirable cell distribution (1-10, 1-20, 1-30, 2-10, 2-20, 2-30 cells per well etc.) to ensure that all or the majority of cells/nuclei distribution are captured, while still enabling capping of the cells in the well with the bead.

In some embodiments, the walls of the microwells may comprise a ridge or baffle, that prevents the beads from coming into contact with the bottom of the wells. As illustrated, the design of the microwells is such that a bead will sink in the microwells and then stop at a construction, when it can go no further. At this point, the outside surface of a bead should be at least partially in contact with the walls of the well, thereby compartmentalizing the volume at the bottom of the well. This concept is shown in FIG. 2A, where V is the compartmentalized volume. FIG. 2B illustrates a microplate that is made up of cone-shaped wells, where the diameter of the opening of the well is approximately 3× the diameter of the bottom of the well.

FIGS. 3B-3E show various macroplate configurations. As shown, the footprint of a macroplate can be the same as a standard microtiter plate. The macrowell (MaW) can be any size, any shape, and at any position in the macroplate. For example, in some embodiments, the macrowell can be less than the size of a typical well of a 96-well plate. However, in many embodiments, the smallest macrowell may be in the range of 0.5 to 1 cm in diameter. In these embodiments, there may be a minimum of 10,000-10,000 microwells (up to 5-50M microwells) exposed at the bottom of the macrowell. As illustrated, the largest macrowell may occupy most of the microplate. In these embodiments, millions of microwells are exposed at the bottom of the macrowell.

In some embodiments, the microwell plate may be larger than the size of a typical 96-well microtiter plate. Increasing the dimensions of the microwell plate should lead to a proportional increase in the number of cells that can be processed in a given assay. In these embodiments, the total plate area may be 1 to 2 times larger than a standard 96-well microtiter plate, or 2 to 4 times larger than a standard 96-well microtiter plate, or 4 to 10 times larger than a standard 96-well microtiter plate, or more than 10 times larger than a standard 96-well microtiter plate.

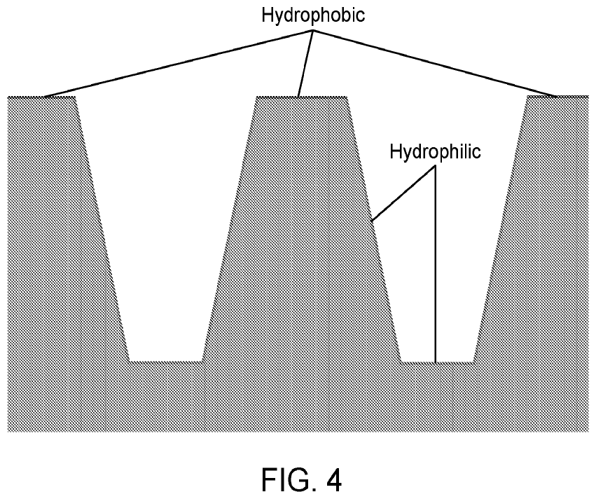

In some embodiments, the top of the microwell plate but not the walls of the wells of the microwell plate may be hydrophobic and/or the walls of the wells of the microwell plate but not the top of the microwell plate may be hydrophilic. This concept is illustrated in FIG. 4. These coatings can be added after fabrication of the plate, or in some case, the plate can be fabricated from a material that already has the desired surface properties. As noted above, these surfaces may aid in distributing liquid that has been deposited onto the top surface of the microplate into the micro-wells. In some embodiments, this plate can optically clear and/or have low background fluorescence to inspect fluorescently marked cells and/or nuclei in the wells. In some embodiments, the microplate could be made from a biologically inert material, particularly a material that that does not significantly bind DNA or protein such as COP (cyclo olefin polymer), but other polymers could be used instead. In some embodiments, the microwell plate can be made of glass or other transparent materials to enable imaging (morphology, fluorescence, contrast microscopy etc.) of cells from the bottom of the plate.

### Kits

The present assembly may be used in conjunction with beads of a defined diameter. As noted above, wherein in some embodiments the wells of the microwell plate are dimensioned to allow a single bead into the well but comprise a constriction that prevents the bead from coming into contact with the bottom of the well. Kits that contain an assembly and the beads are therefore provided. The various components of the kit may be provided in separate containers, and, in some embodiments, the kit may have instructions for performing the method set forth below. In some embodiments, the beads may be each uniquely barcoded by oligonucleotides that are tethered to the beads. Such beads can be made by a variety of specific methods, including the split-pool method described in U.S. Pat. No. 11,692,214. Such beads have oligonucleotides tethered to them, where the tethered oligonucleotides on each bead have a unique sequence that is different to the sequence that is in the oligonucleotides that are tethered to other beads. In other words, if there are 1,000 barcoded beads, the oligonucleotides that are tethered to each bead will have a unique barcode sequence (referred to herein as a “second index sequence). The index sequence for one bead is different to the index sequences for other beads. As such, this index sequence is “bead-specific”. In addition to the bead barcode sequence, optional UMI can also be included on the bead tethered oligonucleotide. The UMI uniquely labels each molecule interacting with a given barcoded bead facilitating accurate digital counting of assay molecules. In some embodiments, the barcoded beads contain a capture oligonucleotide sequence for capturing target molecules from a cell. In the desired configuration, the capture sequence is designed to capture barcoded cellular target molecules from cells either directly or through a splint oligonucleotide. In some embodiments, the barcoded bead contains two or more unique capture sequences for capturing two or more unique target molecules of the cell. For example, two different mRNA/cDNA molecules, two different DNA molecules, or different classes of analytes including DNA, RNA, and protein or any combination thereof.

A kit may contain at least 10,000, at least 100,000, at least a million, at least 10 million, at least 100 million or at least a billion barcoded beads (each having a unique index sequence). The beads should be spherical, although other shaped beads could also be used. As discussed above, in some embodiment, the beads may have a diameter that permits them to enter the microwells but not reach the bottom of the microwells. In many embodiments, the particles have a diameter in the range of 10-200 microns, e.g., 20-100 microns, 20-40 microns, 40-60 microns, 60-80 microns or 80-100 microns. As would be apparent, the diameter of the beads can be 20-80% (e.g., 20-60% or 40-80%) of the diameter of the entrance to the wells (allowing the beads to enter the wells and sink into the wells) and the diameter of the beads may be greater (e.g., by 20-80%, 20-60% or 40-80%) than the diameter of the well at the bottom of the wells (which prevents the beads from sinking to the far end of the wells).

In some embodiments, the barcoded beads may be made by emulsion PCR, which method has been successfully used for other applications and is described in, e.g., Kanagal-Shamanna et al (Methods Mol Biol 2016 1392:33-42) and Shao et al (PlosOne 2011 0024910). In some embodiments, the method may involve coating a population of beads with a forward primer (e.g., via click chemistry, streptavidin, or via a covalent interaction), combining the beads with a reverse primer, dNTPs, polymerase and an oligonucleotide template that has a 5′ sequence that hybridizes with the forward primer, a variable, e.g., random, sequence that produces an index sequence when copied and a 3′ sequence corresponding to the reverse primer, producing an emulsion, where each droplet contains on average a single bead, a single molecule of template, and multiple molecules of reverse primer, and thermocycling the emulsion, thereby grafting copies of the sequence of the template onto the forward primers. The strand that is not required can be subsequently removed by denaturation. As would be understood, the template may have a forward primer binding site, a degenerate (e.g., random) sequence of 6-10 nucleotides (or even more random nucleotides dependent on the number of unique sequences required) and sequence that provides a binding site for the reverse primer, when it is copied.

In some embodiment, the barcode beads may be made by split-pool synthesis such as that described by Delley et al. (Sci Rep 2021 11, 10857) in which barcodes are generated by enzymatic step-wise addition of oligonucleotide sub-barcode words to a nascent oligonucleotide sequence. The number of possible unique barcode beads generated is dependent on the number of sub-barcodes employed at each step and the number of steps. For instance, a set of 96 sub-barcodes per step X 4 steps generates a total of 96×96×96×96=almost 85 million unique barcode bead types. Random assembly of this bead set in a 1 million feature microwell substrate will minimize barcode collisions given the statistics of choosing 1 million beads from a pool of 85 million bead types (birthday problem statistics). The statistics of the “birthday problem” are summarized by the following equation (Li et al, Kidney 2021 360 2:1196-1204):

$$P = \frac{N - D + D {( \frac{D - 1}{D} )}^{N}}{N} \sim \frac{N}{2 D} for large N and D$$

where P=collision rate; N=number of microwells; and D=number of unique barcode bead types. For D tenfold greater than N, P~5%. In general, when barcoded bead types are in excess of microwells by ten-fold, there is roughly a 5% collision rate; and when in excess of microwells by hundred-fold there is less than 0.5% collision rate.

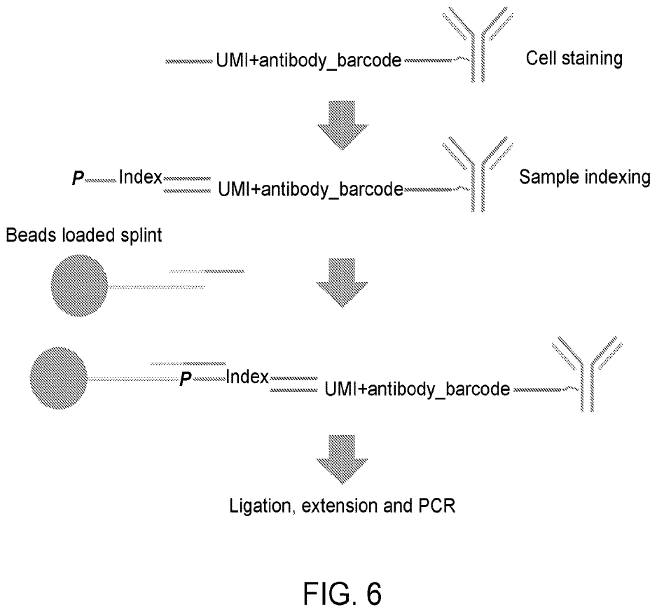

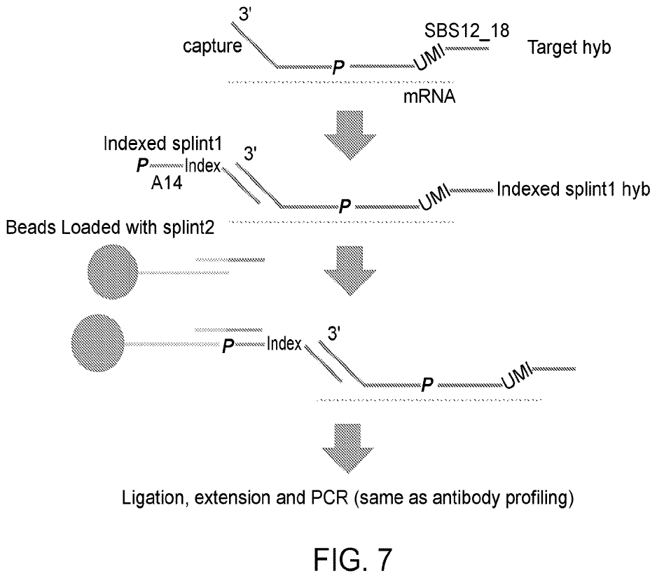

In some embodiments, the beads or the pre-barcoded microwell may be pre-loaded with a barcoded splint, as illustrated in FIGS. 6 and 7. A splint is used in assay formats that employ ligation, particularly when ligating the 5′ ends of barcoded target DNA sequences.

In some embodiments, the beads or the pre-barcoded microwell may be pre-loaded with a biotinylated oligonucleotide of complementary sequence to a portion of the oligonucleotide present on the barcoded microwell or bead. In some embodiments, the biotinylated oligonucleotide may be pre-functionalized with a streptavidin protein. In some embodiments, there may be an biotin moiety added to target molecules of cells prior to lysis. In some embodiments the biotinylated, either directly or indirectly, target molecules may be captured onto the streptavidin functionalized bead. In some embodiments, the oligonucleotide can function as a hybridization capture sequence for target molecules. In some embodiments, the biotin-streptavidin interaction enhances the capture of the target molecules on beads. In some embodiments, the interaction between the modified target molecule and modified bead can be other than biotin-streptavidin (e.g. chemical, click chemistry, antibody-antigen, etc). In some embodiments, the capture of the target molecule with the bead is accelerated and/or the efficiency is improved using methods other than DNA hybridization.

In some embodiments, the beads or microspheres may be porous and/or may contain one or more channels. Cellulose porous beads (CPBs), agarose porous beads (e.g. Sepharose), porous polystyrene/divinyl benzene (PS/DVB) beads, porous silica beads (Eprui Biotech, Shanghai, China), other types of porous beads could be employed although there are many alternatives (see, e.g., Kai et al AJNR Am J Neuroradiol. 2006 27:1146-50 and Pu et al Environ Sci Pollut Res Int. 2017 24:16520-16530). In some embodiments, the beads are magnetic to facilitate downstream assay workflows either on the microwell plate or after removing the beads from the microwell plate. In some embodiments, the beads are functionalized with oligonucleotides and universal capture sequence. A capture sequence can bind to analytes, hybridize to analytes, or capture pre-barcoded analytes or derivatives. In some embodiments, the bead can be dissolved or disintegrated using solvents or reagents.

A kit may further comprise other reagents that are described in more detail in the methods section below. For example, a kit may comprise a set of at least 10 (e.g., at least 8, at least 16, at least 48, at least 96 oligonucleotides, or at least 384 oligonucleotides (e.g., primers) that have different barcodes. In some of these embodiments, the oligonucleotides may a 3′ end that has a random, oligo-(dT), or gene-specific sequence, and a barcode (or “index”) that is 5′ of that sequence. These oligonucleotides will typically be in separate containers. These oligonucleotides can be used as reverse transcription primers in the method described below. Alternatively, the oligonucleotides may be ligated onto adapter sequences associated with the analytes. In addition, a kit may contain a container of splint oligonucleotides, overlay oil, overlay of viscous or diffusion limiting solution, cell lysis buffer containing diffusion limiting reagents (e.g. PEG, glycerol etc., see FIG. 13), a cell lysis buffer and/or a collection buffer, etc., as described below. An overlay over the microwells can be used to confine analytes to the microwells. In some cases, the overlay improves capture of analytes within the microwell or bead. The overlay can be an oil, an immiscible material, a material that restricts or reduces diffusion, a composition of 2 or more materials, or enables reagent exchange. The overlay can be a liquid (e.g. oil) or a solid material (e.g. agarose). In some cases, the solid materials can be changed into a liquid form and removed from the microwells (e.g. low melting agarose, dissolve etc). In some cases, the material is solubilized using solvents or liquids. In some embodiments, the overlay is reversible.

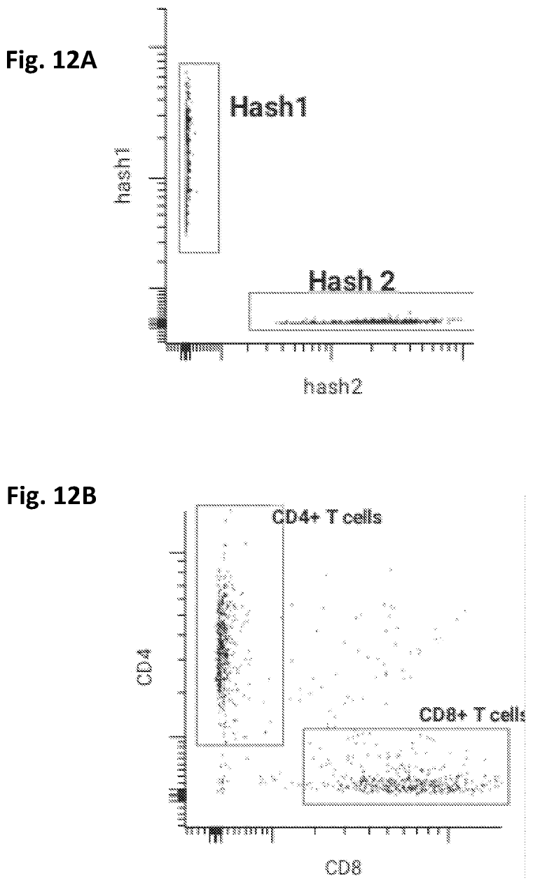

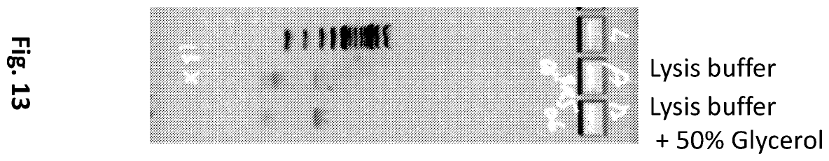

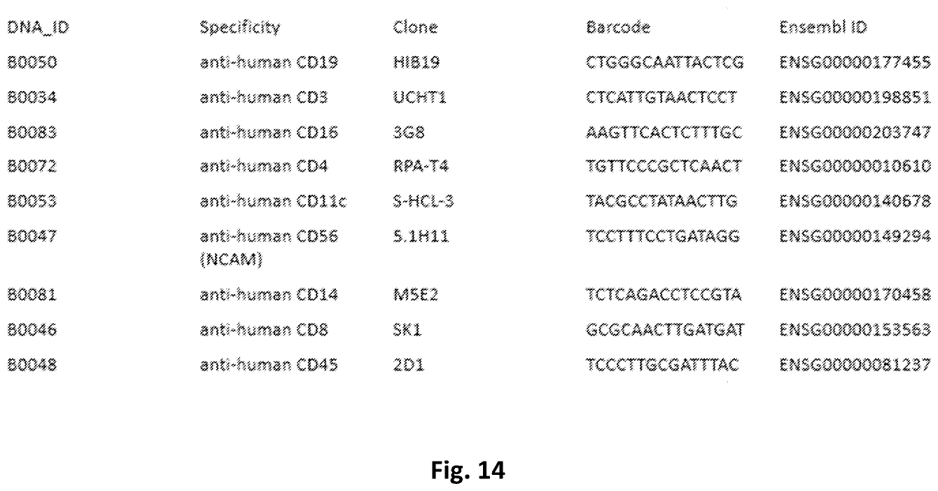

The methods and compositions described can be used or combined with cell hashing. In one embodiment, cell hashing uses oligo-tagged antibodies against surface proteins to place a “sample barcode” on each single cell, enabling different samples to be multiplexed together and run in a single experiment (FIG. 12A). See, e.g., Stoeckius et al. Genome Biology 2018 19:224.

### Methods

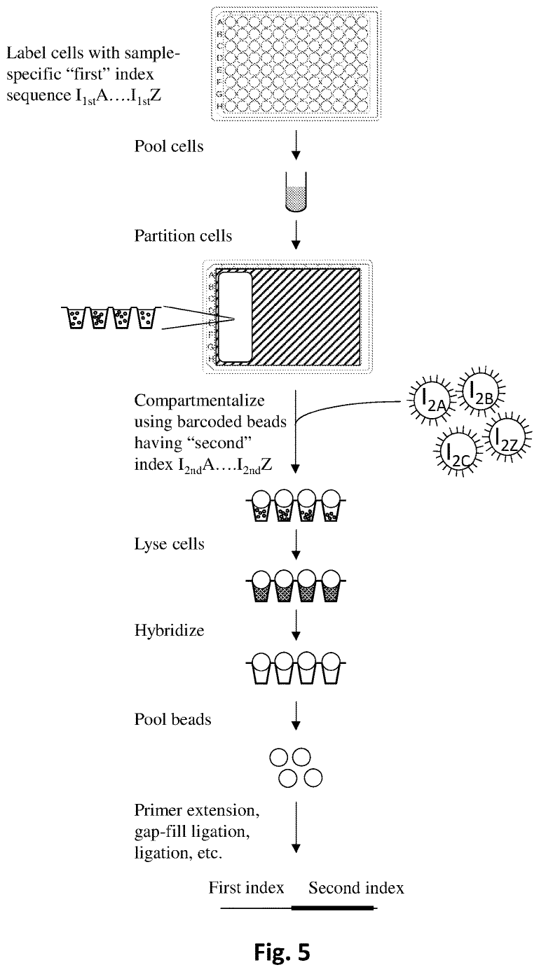

A method that may employ the above-described assembly is provided. In some embodiments, the method is for barcoding nucleic acids that are associated with biological particles (e.g., cells or nuclei or cell organelles isolated from cells). In some embodiments, this method may comprise: (a) obtaining a set of compartmentalized samples in which each compartment comprise multiple biological particles, wherein the biological particles comprise nucleic acids that are associated with a first index sequence that is different in each of the compartments (i.e., a “compartment-specific” index sequence); (b) combining the barcoded samples into a pool, thereby generating a pool of biological particles; (c) partitioning the pool of biological particles into the wells of a microwell plate, wherein each well of the microwell plate that receives a biological particle receives an average of at least 1 or more or at least 2 biological particles; (d) compartmentalizing the biological particles in the wells by distributing a population of beads across the wells, wherein: each well receives, on average, a single bead; the beads enter the wells but do not come into contact with the bottom of the wells, thereby encapsulating the particles in compartments; and the beads are uniquely barcoded by oligonucleotides that are tethered to the beads and comprise a second index sequence that is different for each of the beads (i.e., a “bead-specific” index sequence); (e) lysing the biological particles while they are in the compartments to release the nucleic acids that comprise the first index sequences into solution; (f) incubating the microwell plate under conditions sufficient for the released nucleic acids to form complexes with the barcoded oligonucleotides that are tethered to the beads in the compartments, to produce complexes comprising a first index sequence and a second index sequence (these complexes are tethered to the beads); (g) pooling the beads; and (h) performing an en masse molecular reaction on the beads to produce reaction products that contain a first index sequence or complement thereof and a second index sequence or complement thereof. An example of this method is schematically illustrated in FIG. 5.

In some embodiments, the microwells are overlayed with a semi-permeable gel or membrane prior to cell lysis. The semi-permeable overlay allows lysis reagents, such as salts and detergents, to easily diffuse into the microwell but minimizes the diffusion of higher molecular weight molecules out of the microwell. This format enhances the capture of the DNA tagged cellular analytes on the compartmentalized barcode bead. In some embodiments, the semi-permeable material is an agarose or polyacrylamide gel overlay. In some embodiments, the agarose gel overlay is a low melting point (LMP) agarose gel. In some embodiments, the LMP agarose gel is dissolved post-lysis/annealing by using heat and/or agarose enzyme to dissolve the LMP gel. In some embodiments, the polyacrylamide gel is comprised of disulfide crosslinks which enable easy dissolution post-lysis/annealing using a reducing agent such as dithiothreitol (DTT), beta-mercaptoethanol, TCEP, etc.

A method that may employ the above-described assembly is provided. In some embodiments, the method is for barcoding nucleic acids that are associated with biological particles (e.g., cells or nuclei isolated from cells). In some embodiments, this method may comprise: (a) obtaining a set of compartmentalized samples in which each compartment comprise multiple biological particles, wherein the biological particles comprise nucleic acids that are associated with a first index sequence that is different in each of the compartments (i.e., a “compartment-specific” index sequence); (b) combining the barcoded samples into a pool, thereby generating a pool of biological particles; (c) partitioning the pool of biological particles into the wells of a microwell plate, wherein each well of the microwell plate that receives a biological particle receives an average of at least 1 or more biological particles; (d) lysing the biological particles while they are in the compartments to release the nucleic acids that comprise the first index sequences into solution; (e) compartmentalizing the biological particles in the wells by flowing a layer of oil across the wells, thereby encapsulating the particles in compartments; (f) incubating the microwell plate under conditions sufficient for the released nucleic acids to form complexes with the a second index comprised of barcoded oligonucleotides that are tethered directly to the compartments (the second index sequence is different for each of the microwells (i.e., a “microwell-specific” index sequence); to produce complexes comprising a first index sequence and a second index sequence enabling the analytes of the particles to be uniquely barcoded, and (g) performing an en masse molecular reaction on the microwells to produce reaction products that contain a first index sequence or complement thereof and a second index sequence or complement thereof. An example of this method is schematically illustrated in FIG. 5. Finally, cleaving/eluting all completed complexes from the microwells into a final pool of library elements. (PCR or emulsion PCR)

In step (a) of the method, the set of compartmentalized samples may contain at least 1, at least 2, at least 4, at least 12, at least 48, at least 96 or at least 384 different samples that each comprise at least 100, at least 500, at least 1,000, at least 5,000, at least 10,000, at least 50,000 or at least 100,000 biological particles (e.g., cells or nuclei isolated from cells). As indicated above, the biological particles comprise nucleic acids that are associated with a first index sequence that is different in each of the compartments (i.e., a “compartment-specific” index sequence). In other words, the samples are barcoded with a sample identifier sequence, i.e., the particles in each compartment are associated with an index sequence that differs from compartment to compartment. In some embodiments, the compartmentalized samples may contain biological particles that have not been previously split. In these embodiments, the different samples may be from different individuals, samples taken at different time points, or samples taken from different tissues, where the first index sequence is sample-specific (i.e., the index sequence differs from sample to sample and may identify the patient from which the sample was obtained, etc.). Such barcodes are referred to as “sample-specific” barcodes herein. In an alternative embodiment, the compartmentalized samples may contain biological particles that have been barcoded and then split. In these alternative embodiments, an initial biological sample may have been split into the compartments. In these embodiments, more than one or all of the biological particles in the compartments may be derived from the same sample.

The first index sequence may be added to the biological particles in the sample by any of a number of different methods, e.g., by hybridization, ligation, a gap-fill/ligation and/or primer extension. For example, in some embodiments, the first index sequence may be in a reverse transcription primer. In these embodiments, the first index sequence may be added in a reverse transcription reaction to produce indexed cDNA in the cells, which may be done in situ on fixed, permeabilized cells (see, e.g., Kuchina et al (Science 2021 371: eaba5257), O'Huallachain et al (Commun. Biol. 2020 3:279), Cao et al (Science 2017 357:661-667), Rosenberg (Science 2018 360:176-182), WO2019236599, and WO2016100976, among others. In these embodiments, the samples may be reverse transcribed in the compartments using different reverse transcription primers (where the different primers have different index sequences). The 3′ end of these primers may have an oligo (dT) sequence or a random sequence. In some embodiments, the 3′ end of these primers may be gene-specific. In other embodiments, the first index sequence may be added to the oligonucleotide component of one or more antibody-oligonucleotide conjugates by hybridization, ligation, primer extension or a gap-fill/ligation reaction, where the antibody-oligonucleotide conjugates have previously been bound to the particles (see, e.g., WO2012106385, WO2014026032 and WO2016100976). Similar methods may be used to add first index sequences to other types of nucleic acids that may be pre-made in or on the particles (e.g., fragments of genomic DNA, ligation products, tagged genomic DNA, proximity assay products, tagmented genomic DNA, hybridization products, etc.) as desired (see, e.g., WO2016100976). If the index sequence is ligated onto the nucleic acid, then the ligation can be a splinted ligation, but other methods can be used. In some embodiments, the first index sequence may be added by hybridization (see, e.g., FIGS. 6 and 7). As illustrated in FIG. 5, the first indexes can be added to biological particles that are in a multi-well plate, where each well receives a different first index sequence (i.e., a different I₁ₛₜ sequence). In some embodiments, the nucleic acid that contains the first index sequence may also contain a unique molecular index or UMI, which may be a random sequence of nucleotides (of a length of, e.g., 4-30 nucleotides). Alternatively, the nucleic acid added in this step of the method may be added to another nucleic acid that has a UMI.

In the next step of the method, the barcoded samples are pooled, i.e., mixed together, to generate a pool of biological particles. This may be done by pipetting each of the samples into the same vessel or by inverting the plate that contains the compartmentalized samples into a collection plate and then gently centrifuging the sample into the collection plate.

After the barcoded samples have been pooled, the method involves partitioning the pool of biological particles into the wells of a microwell plate, wherein each well of the microwell plate that receives a biological particle receives an average of at least one or more biological particles (e.g., an average of 2-3 biological particles for 96 indexes and 384 indexes respectively for a 1% collision rate/doublet rate or so). The number of cells/nuclei that can be loaded per well and still enable single cell resolution is dictated by the number of first barcodes applied. With 100 first barcodes and two cells per microwell loaded, the chance of having cells assigned the same barcode combination is 2%. In some embodiments, a single cell or nuclei is loaded per microwell while occupying the majority of microwells with a cell or nuclei. Microwells can have various dimensions accommodating a range of cells/nuclei. Cells/nuclei are randomly distributed across the microwells resulting in a Poisson distribution of cells across the microwells. The microwells need to be large enough to accommodate this distribution while still enabling the loading of the beads in the microwells. This ensures that all cells are captured and processed into single cell libraries. In some cases, the microwell plate is preincubated or washed with buffers, salts, pluronic acid, ethanol, BSA etc. to facilitate or improve cell loading. In some cases, the present assembly can be used in this step (as illustrated in FIG. 5). In these embodiments, if the total number of cells in the pool is known then the appropriate size of macrowell can be chosen (see FIG. 3). In these embodiments, the pool of cells may be placed on top of the exposed microwells and the cells and medium may enter the microwells by gravity or gentle centrifugation. Appropriate hydrophobic/hydrophilic surfaces may facilitate dispersal of the cells across the plate and into the wells.

The number of microwells selected for this part of the method (which, in turn, determines the size of the macrowell) can be calculated, if necessary, so that less than 5%, less than 2%, less than 1%, less than 0.5% or less than 0.1% of the microwells receive two particles that are associated with the same first index sequence. Assuming that the distribution of the biological particles into the microwells is random, then the occupancy of biological particles that have the same first index sequence in the wells should have a Poisson distribution. Thus, if there are 10,000 biological particles that have the same first index sequence in a pool, the minimal number of microwells required to ensure that at least 9,990 (or whatever number is desired) of the biological particles that have the same index go into different microwells (i.e., are “singletons”) can be readily calculated.

After the biological particles have been partitioned into the wells of the microplate, the biological particles are compartmentalized using the barcoded beads, which at least partially enter the microwells (one per well) and sink until they lodge against the constriction and can move no further. These beads effectively block the exits to the microwells, preventing the cells from moving from one well to the other. As noted above, the wells should be dimensioned to each receive, on average, a single bead, and the beads enter the wells but do not come into contact with the bottom of the wells. As described above, oligonucleotides comprised of a second index sequence are tethered to the beads. In these embodiments, the second index sequence is bead-specific in the sense that each bead is associated with a different index sequence.

Next, the biological particles are lysed while they are in the compartments. This step releases into solution the nucleic acids that are associated with the first index sequence. This step may be done by a variety of different methods. In some embodiments, the microplate may be heated or an external stimulus (e.g., microwaves) may be applied. In other embodiments, a dried lysis reagent (e.g., a surfactant) that has been pre-dried onto the bottom of the microwells before the biological particles have been added may be released and/or dissolved via an external stimulus. In some embodiments, the lysis is initiated by an external stimulus including, but not limited, light or temperature. In some embodiments, the lysis is performed with diffusion limiting components, e.g., glycerol (see FIG. 13). In some embodiments, the beads are non-porous, and diffusion of reagents occurs through the interface of the bead and the microwell. In some embodiments, the micowells have ‘channels’, microstructures, or openings that facilitate the transfer of reagents to the inside of the microwell. In alternative embodiments, the beads may be porous or may contain channels, in which case a lysis solution (e.g., a surfactant) may be deposited onto the top surface of the microplate and the surfactant may diffuse through the beads to the compartmentalized biological particles. If this approach is used, then the surfactant may be combined with an oil layer (e.g., mineral oil) to “overlay” the microwells and prevent any well-to-well contamination. In some embodiments, the bead provides the second barcode but does not help or contribute the confinement of the cellular materials inside the microwell. In some cases, the overlay as described in this application is solely responsible for the containment of the cellular materials inside the microwell.

After the cells are lysed, the microwell plate (or the entire assembly) may be incubated under conditions sufficient for the released nucleic acids to form complexes, i.e., hybridize, with the oligonucleotides that are tethered to the beads in the compartments or hybridize indirectly to the beads using a splint oligonucleotide. The bead or the target nucleic acids contain a splint oligonucleotide to facilitate hybridization to the bead. In some embodiments, the lysis buffer contains reagents that limit diffusion, for example glycerol, dextran sulphate, or PEG. This step may be done by incubating the assembly at a temperature in the range of room temperature to 37 °C to 65 °C for a period of 5 mins to 12 hours. As would be apparent, the medium in which the biological particles were deposited onto the microplate should contain a sufficient amount of salt for hybridization to occur. Since this medium was used in the initial step of the method (i.e., to add the first barcodes to the samples) the medium should not need to be modified. This step results in beads that have complexes comprising the first index sequence and the second index sequence. These complexes are tethered to the beads. As would be apparent, the ends of the nucleic acids that add the first and second index sequences can be designed to be complementary, so that one can be extended using the other as a template. Alternatively, a splint oligonucleotide could be used to splint the ends of those nucleic acids together so that one can be ligated to the other, for example.

After the released nucleic acids have formed complexes with the oligonucleotides that are tethered to the beads, the beads are then pooled. This may be done by placing a collection plate over the microplate (or assembly, if used) and forcing the beads into the collection plate by centrifugal force. Other methods (e.g., electromagnetism) may also be used, particularly if the beads are paramagnetic (e.g. magnetic pin). In some embodiments, the beads arc pooled/collected using a high-density solution using buoyancy or upthrust (e.g. NaCl, Tungsten acetate, tungsten chloride, sucrose etc.).

After the beads are collected, an en masse molecular reaction is performed on the beads to produce reaction products that contain a first index sequence or complement thereof and a second index sequence or complement thereof. In this step, the collected beads may be optionally washed and then an enzyme mix that contains ligase and/or a polymerase (and any other necessary components such as dNTPs) may be added to the beads to produce a reaction mix, and the reaction mix is incubated under conditions suitable to produce reaction products that contain the first index sequence or complement thereof and the second index sequence or complement thereof. As noted above, this reaction may be a ligation (which may be splinted), a gap-fill ligation or a primer extension reaction, for example. In some embodiments, the reaction may be a ligation followed by an extension. The reaction product should have a strand that contains a first index sequence or complement thereof and a second index sequence or complement thereof, which strand can be optionally cleaved from the beads and then amplified and sequenced. In some embodiments, the nucleic acids added in the method may contain primer binding sites and, as such, in some embodiments, the method may comprise sequencing the reaction products, or an amplification product thereof, to produce sequence reads. The reaction product may undergo other further manipulations at this point, e.g., it could be made double-stranded, tagmented, nicked, or cleaved, etc.

In some embodiments, the microwells are overlayed with a semi-permeable gel or membrane prior to cell lysis. The semi-permeable overlay allows lysis reagents such as salts and detergents to easily diffuse into the microwell but minimizes the diffusion of higher molecular weight molecules out of the microwell enhancing capture of the DNA tagged cellular analytes on the compartmentalized barcode bead. In some embodiments, the semi-permeable material is an agarose or polyacrylamide gel overlay. In some embodiments, the agarose gel overlay is a low melting point (LMP) agarose gel. Example of an agarose overlay is described in Wood, D. K., et al. (2010) Single cell trapping and DNA damage analysis using microwell arrays. Proceedings of the National Academy of Sciences of the United States of America, 107 (22), 10008-10013. In some embodiments, the LMP agarose gel is dissolved post-lysis/annealing by using heat and/or agarose enzyme to dissolve the LMP gel. In some embodiments, the polyacrylamide gel is comprised of disulfide crosslinks which enable easy dissolution post-lysis/annealing using a reducing agent such as dithiothreitol (DTT), beta-mercaptoethanol, TCEP, etc.

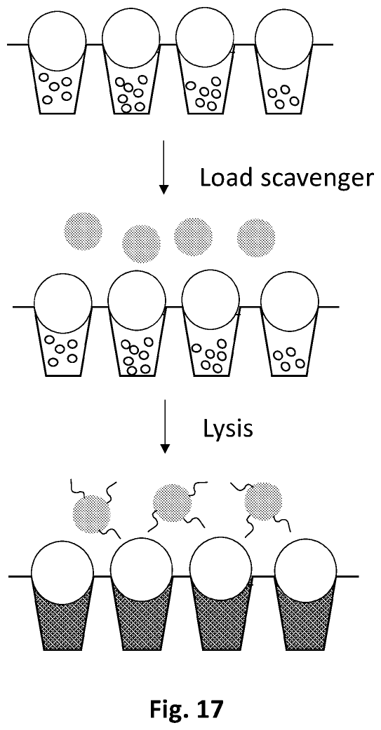

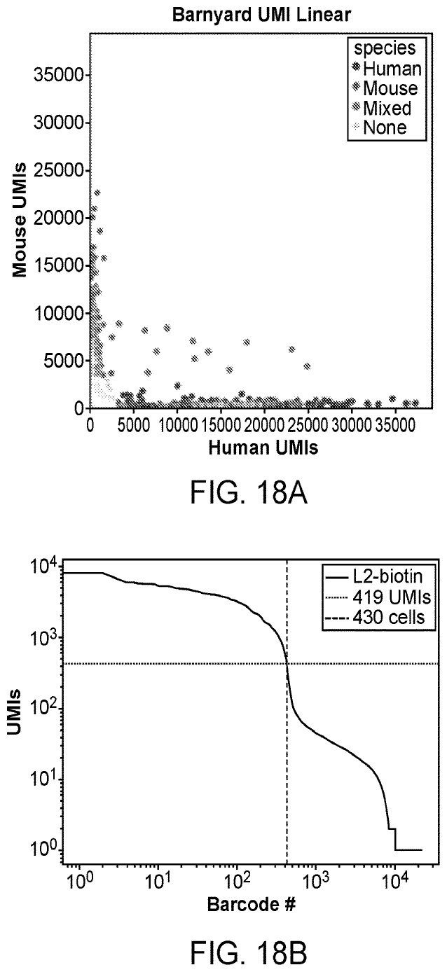

In some embodiments, a scavenger is added (FIG. 17). The function of a scavenger is to ensure no or a minimal number of analytes from cells are captured by beads other than from the well and bead the cells originally reside in before lysis. In some embodiments, the scavenger is an oligonucleotide for capturing target molecules either on a solid support (e.g., bead) or in solution. The scavenger and associated target molecules can be either removed before generating the sequencing library, or not removed prior to library generation.

Alternatively to the use of beads, pre-barcoded microwell arrays can be employed, and, after the biological particles have been partitioned into the wells of the microplate, the biological particles are compartmentalized by using an overlay of oil across the microplate such as light mineral oil, or heavier fluorinated oil (e.g. Fluorinert FC-40) or perfluorinated oil (F3556-25ML, Sigma-Aldrich) or similar oil (see Baret, Lab on a Chip, 2012 12:422-433). In a preferred embodiment, the oil overlay is performed in a microfluidics device, such as described by Yuan and Sims (2016) that allows rapid fluid exchanges. An alternate method is to overlay the microwell array is to use a molecular weight cutoff membrane (MWCO) placed across the microwell array such that only small molecules can diffuse through the membrane retaining cells, proteins, and nucleic acids. The oil or membrane overlay effectively blocks the exits to the microwells, preventing the biological particles from moving from one well to the other. After compartmentalization with oil or the MWCO membrane, the biological particles are lysed and any optionally crosslinkers reversed using a reducing agent. In the case of oil overlay compartmentalization, lysis can be accomplished by photoactivation of a photo-activatable detergent or surfactant, or via pH changes using acids and bases soluble in the oil overlay which enables acidification or alkalization of the aqueous phase in the microwells (Mashagi et al, Scientific Reports, 2015 5, 118). Additionally, an oil soluble reducing agent, such as 2-(Dibenzylamino)butane-1,4-dithiol (DABDT) can also be use in the oil overlay to provide a reducing agent to the aqueous phase in the microwell (Mthembu et al., Organic Letters, 2019 21:10111-10114). In some embodiments, the lysis of cells inside microwells is performed using freezing (Dura et. Al, Nucleic Acids Res., 2019 47: e16).

FIGS. 6 and 7 illustrate two molecular biology workflows that could be employed in the present method. FIG. 6 illustrates a workflow for the analysis of proteins and FIG. 7 illustrates a workflow for analysis of mRNA. Both workflows represent examples only. Given these examples, one could readily figure out other methods and/or adapt the methods for the analysis of tagmentation patterns in chromatin, genome analysis, reverse transcription products, ligation products, and hybridization products, etc.

In FIG. 6, cells or nuclei are stained with one or more antibody-oligonucleotide conjugates that contain an oligonucleotide that has a random sequence (a UMI) and a barcode sequence that indicates that identity of the antibody to which it is joined. The staining may be done in the compartments of step (a). Alternatively, cells or nuclei may be transferred to the compartments after staining. In the first step of this method (prior to pooling), oligonucleotides that contain sample-specific indexes are added to the compartments, and the oligonucleotides hybridize with the oligonucleotides of the conjugates. This indexes or “tags” the sample with a sequence that identifies the sample. In this workflow, the barcoded beads may be pre-loaded with a splint oligonucleotide that hybridizes with the other end of the oligonucleotides that contain sample-specific indexes (i.e., the end that is not hybridized to the oligonucleotide of the antibody-oligonucleotide conjugate), as shown in FIG. 6. In this implementation the two indexes can be combined into a single strand via a ligation followed by an extension. This molecule can be amplified and then sequenced.

In FIG. 7, mRNA in cells or nuclei are hybridized with pairs of oligonucleotides that hybridize to adjacent sites. In this implementation, the cells or nuclei are hybridized to the pairs of oligonucleotides, and any oligonucleotides that hybridize to adjacent sites are ligated together. As shown, one of the oligonucleotides may contain a random sequence (a UMI). This reaction may be done while the cells or nuclei are in the compartments of step (a). Alternatively, the cells or nuclei may be transferred to compartments after the reaction. The remainder of the is method is similar to the method illustrated in FIG. 6.

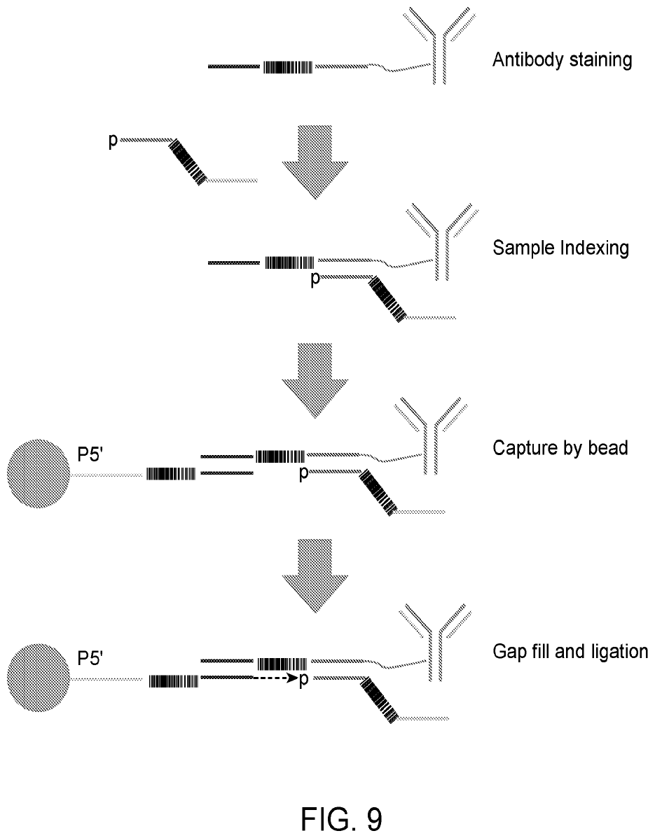

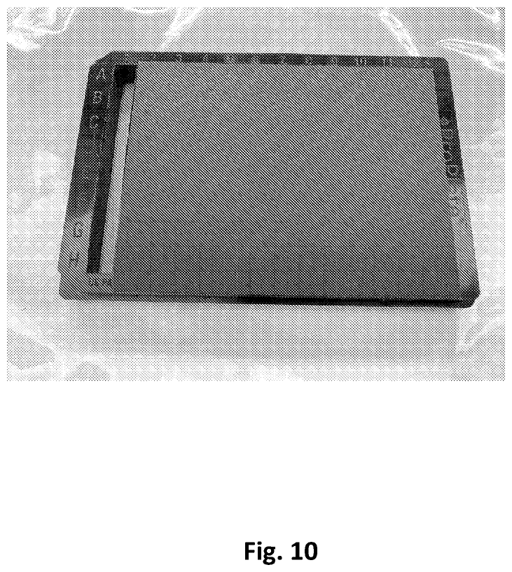

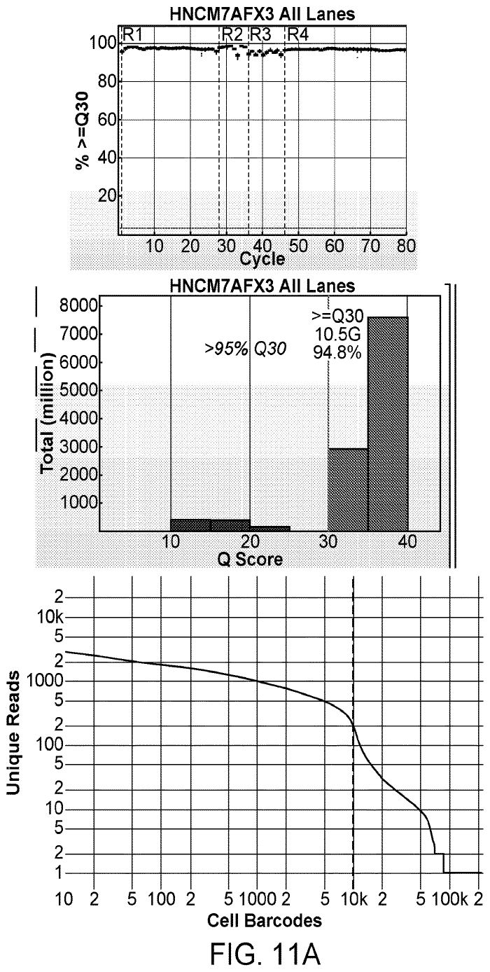

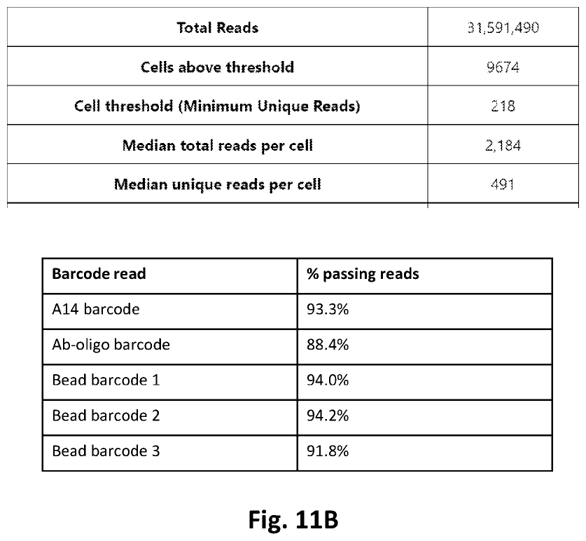

FIG. 9 illustrates a workflow for the detection of protein wherein the assembly is directly captured on a bead without the need of a splint oligonucleotide. Advantages include a smaller number of washing steps, and maintaining single strand probes on the bead that, at a later step, can be removed by for example an exonuclease.

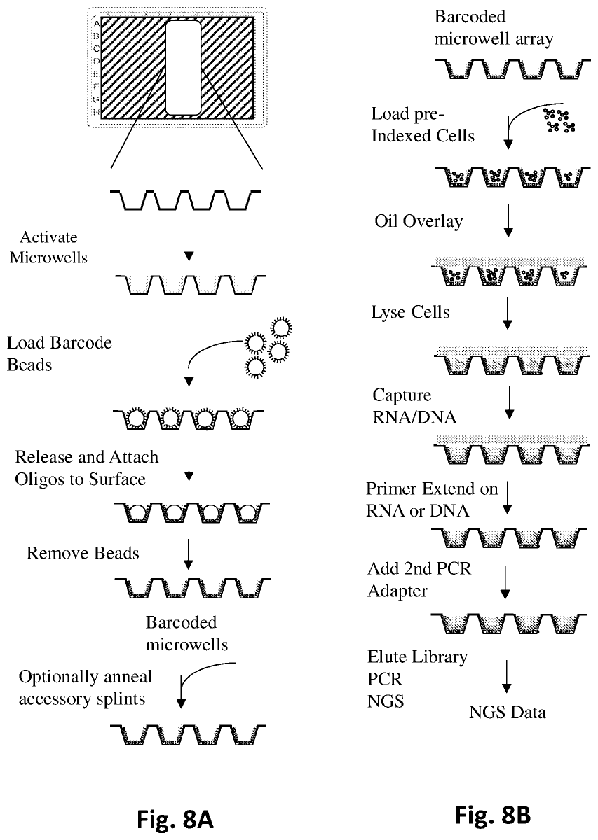

FIG. 8 illustrates an alternative embodiment that results in a microplate that comprises barcoded wells, i.e., at least 1,000, at least 5,000, at least 10,000, at least 100,000, at least 500,000, at least 1M, at least 5M barcoded wells. As illustrated in (A), this may be done by activating the microwells, adding barcoded beads such that the majority of the wells receives a single bead, releasing the oligonucleotides from the beads and allowing the released oligonucleotides to become tethered to the surface of the wells. The beads can then be removed and, if necessary, a universal splint can be added to the wells. (B) illustrates how the “barcoded microwell” plate can be used for single cells analysis.

In some embodiments, this method may be implanted by a method comprising: (a) obtaining: an activated microwell array comprising wells that have diameters that are in the range of 30-100 microns in diameter; barcoded beads with a diameter matched to the diameter of the microwell array such that only a single bead can load in any given microwell; and a population of activated and reversibly attached oligonucleotide barcodes on the barcoded beads, (b) assembling the barcoded beads into the microwell array of, wherein a majority e.g., at least 50%, at least 60%, at least 70% or at least 80% of the wells receives a single bead per well; (c) releasing the activated oligonucleotide barcodes from the beads in the wells; (d) coupling of the activated oligonucleotide barcode to the activated microwell surface; and (d) removing the beads from the microwells. The analysis method may comprise: (a) partitioning biological particles (e.g., cells or nuclei) into the microwells of a pre-barcoded microwell array with an average of greater than 1 particle per microwell; (b) lysing contents of biological particles; (c) ligating pre-indexed sample analytes onto pre-barcoded oligonucleotide tethered to microwells; (d) performing primer extension reaction to create a copy of the complete ligated bi-indexed complexes; (e) eluting copy of bi-indexed complexes from microwells and pool; and (f) sequencing the eluted material, or an amplification product thereof.

The product nucleic acids may be sequenced by any suitable system including Illumina's reversible terminator method, Roche's pyrosequencing method (454), Life Technologies' sequencing by ligation (the SOLID platform), Ultima Genomics (e.g. UG100™), singular genomics (e.g. G4 system), element biosciences (e.g. Aviti™ system), Life Technologies' Ion Torrent platform or Pacific Biosciences' fluorescent base-cleavage method and any other platforms e.g. Oxford Nanopore. Examples of such methods are described in the following references: Margulies et al (Nature 2005 437:376-80); Ronaghi et al (Analytical Biochemistry 1996 242:84-9); Shendure (Science 2005 309:1728); Imelfort et al (Brief Bioinform. 2009 10:609-18); Fox ct al (Methods Mol Biol. 2009; 553:79-108); Appleby ct al (Methods Mol Biol. 2009; 513:19-39) English (PLOS One. 2012 7: e47768) and Morozova (Genomics. 2008 92:255-64), which are incorporated by reference for the general descriptions of the methods and the particular steps of the methods, including all starting products, reagents, and final products for each of the steps. The sequencing step may be done using any convenient next generation sequencing method and may result in at least 10,000, at least 100,000, at least 500,000, at least 1M at least 10M at least 100M, at least 1B or at least 10B sequence reads per reaction. In some cases, the reads may be paired-end reads.

After sequencing, the sequence reads that have the same first index sequence or complement thereof and the same second index sequence or complement thereof may be grouped together. In these embodiments, the combination of the first index sequence or complement thereof and the second index sequence or complement thereof identifies a single biological particle (e.g., cell or nuclei) from a particular sample.

In particular embodiments, the assembly may be employed to index analytes in one or more samples using a one-step split and pool approach, the general goal of which is to add a unique index to analytes (e.g., proteins, RNA, DNA, cDNA etc.) that come from the same source (e.g., the same cell or nucleus), so that they can be distinguished from one another. For example, if the sample contains cells, then the index added to the analytes in a particular cell will be different to the index added to the analytes from other cells. In these embodiments, the term “index” is used to refer to a molecule that has a complexity that is sufficient to distinguish between the analytes of the different entities (cells or nuclei).

As noted above, in some embodiments, the sample may comprise cells, nuclei or particles. In these embodiments, the cells or nuclei may be fixed and/or permeabilized prior to starting the method. The sample may contain cells that are in solution, e.g., cultured cells that have been grown as a cell suspension. The sample may contain analytes, (DNA, RNA, and protein) that are uniquely indexed using the split-pool process. In other embodiments, disassociated cells (which cells may have been produced by disassociating cultured cells or cells that are in a solid tissue, e.g., a soft tissue such as liver or spleen, etc. using trypsin or the like) may be used. In particular embodiments, the sample may contain blood cells, e.g., whole blood or a sub-population of cells thereof. Sub-populations of cells in whole blood include platelets, red blood cells (erythrocytes), platelets and white blood cells (i.e., peripheral blood leukocytes, which are made up of neutrophils, lymphocytes, eosinophils, basophils, and monocytes).

### EXAMPLES

To further illustrate some embodiments of the present invention, the following specific examples are given with the understanding that they are being offered to illustrate examples of the present invention and should not be construed in any way as limiting its scope.

### Example 1

### Generation of Pre-Barcoded Microwell Arrays

Barcoded beads are used as a delivery vehicle to deliver a particular unique barcode population to each microwell. The microwells are pre-activated as shown in FIG. 8A, in which the surface of the microwells are activated with a “click chemistry” moiety (e.g. an iEDDA moiety such as TCO, or an SPAAC reactive moiety such as DBCO, or a CuAAC reactive moiety such as alkyne, or a thiol-click moiety or are pre-coated with streptavidin for attachment of capture oligos. The surface can also comprise a 3D polymer surface to increase effective oligo loading. Barcoded beads of proper diameter enable only a single bead to assemble into each microwell on the microwell array. The nucleic acid barcodes on the barcode beads are designed to be removable using a reversible/cleavable linkage (e.g. disulfide bond, photocleavable linker, enzymatically cleavable, pH cleavable, thermolabile, nucleophilically cleavable, oxidatively cleavable, or reductively cleavable). Additionally, the barcode oligos contain a cognate coupling agent capable of reacting with the surface of the activated microwell (e.g. a cognate iEDDA moiety such as mTet, or a cognate SPAAC moiety such as azide, or a cogate CuAAC reactive moiety such as azide, or a biotinylated oligo for streptavidin capture, or a cognate thiol-click moiety. After release of the barcode oligos, they undergo coupling via click chemistry to the activated microwell surface. In particular, iEDDA chemistry is extremely fast and works at even low nM concentration of reactants. After coupling, the beads are removed from the wells via physical or magnetic forces, buoyancy, or via dissolution.

In some embodiments, the microwell array may be subjected to oligonucleotide printing or array-based DNA synthesis to prepare individually barcoded microwells (Large-scale de novo DNA synthesis: technologies and applications Sriram Kosuri & George M Church Nature Methods volume 11, pages 499-507 (2014) DNA synthesis technologies to close the gene writing gap Hoose et al. Nature Reviews Chemistry volume 7, pages 144-161 (2023) and references cited herein)

### Example 2

### Combinatorial Barcoding of Cells with Oligonucleotides Released from Beads or Surfaces

Beads bearing reversible/cleavable combinatorial nucleic acid barcodes are prepared as described above. Cells/Nuclei are fixed and permeabilized and optionally barcoded (for example a barcoded RT primer, barcoded tagmentation etc.) a and a primer (oligonucleotide) bearing a universal annealing handle is hybridized to target molecules in the cell/Nuclei. The cells/Nuclei are then loaded in a non-Poisson limited distribution (superloaded) in a microwell plate containing (2M) microwells, such that multiple cells or Nuclei are present in each microwell.

The oligonucleotide barcodes are then cleaved from the beads via photocleavage mechanism, and diffuse into the cells/Nuclei and hybridize with the universal annealing handle directly or through a splint oligonucleotide. The beads and cells/Nuclei are then recovered from the microwell plate and separated, and the cells/Nuclei have a bulk enzymatic ligation step performed attaching the barcode oligonucleotide to the universal annealing handle. The pooled cells/Nuclei are then split again into a second microwell plate in a non-Poisson limited distribution. A second set of reversible/cleavable combinatorially barcoded beads ‘bearing a barcoded oligo sequence for ligation to the previously introduced barcode oligonucleotide are then loaded. The combinatorial barcodes from the second bead are cleaved, diffuse into the cell/Nuclei, and barcoded ligation provides a second level of indexing, generating (2M×2M=4e12) unique barcodes that can be used for uniquely barcoding 10-100's of millions of cells. Optionally, the second set of barcoded beads are used to capture the first barcoded target molecules after cell lysis, and the second barcode is coupled through ligation on beads.

DNA barcoded microballs can be used instead of barcoded beads. For example, rolling circle amplification can generate clonal DNA barcoded microballs containing multiple copies of a barcoded oligonucleotide. The size of the microball can be tuned through the amplification length of the rolling circle amplification and microball condensation influenced by salts and buffers (Kim et al Biomaterials, 2015, 62:155-63). The size of the microball and microwell can be configured such that only one microball fits into one microwell. Individual copy's of the barcoded oligonucleotides from the microball can be transferred to target molecules or DNA microballs can bind target molecules for barcoding.

### Example 3

### Use of Pre-Barcoded Microwell Array in a 2-Level SCI-Seq Assay

In FIG. 8B, the workflow for using pre-barcoded microwell arrays is shown. A two-level SCI-SEQ assay is implemented by pre-indexing fixed/permeabilized cells using primer extension (RT), affinity binding, or tagmentation with appropriate barcodes primers/adapters. The pre-indexed cells are super-loaded into the microwell arrays such that, on average, multiple cells occupy a given well. The number of multiplicity is determined by the initial complexity of the pre-indexing step and the threshold of cell doublet rate tolerated in the assay. In general, a 96 samples pre-indexing experiment would be practically limited to loading several cells per well (2-5), and a 384 sample set would be limited to 8-20 cells/well. After loading the cells, the loaded microwell array is overlayed with an oil to effectively separate microwells from each other. The cells are lysed using photo-activated detergent that is loaded prior to the oil overlay. Upon UV exposure, the detergent is activated and lysis the cells. The nucleic acids released from the cell hybridize to the barcode surface oligos (probes) via a capture region on the barcode surface probe. This capture region is designed to capture, directly or indirectly (via splint), a region of the adapter index installed onto the nucleic acids during the pre-indexing step. In the case of an RT primer-base indexing, the capture region is a common sequence on the RT primer. After capture of the cellular nucleic acids, the surface barcode primer is extended on or ligated to the cellular pre-indexed nucleic acid target to effectively creating an amplifiable library element.

### Example 4

### Generation of a Barcoded Bead Surface Bearing Additional Capture Probes

A bead bearing an excess of an EDDA reactive click moiety (e.g. methyltetrazine) is prepared. An oligonucleotide bearing the corresponding cognate click moiety (e.g., trans-cyclooctene (TCO)) is brought into contact with bead such that only a fraction (less than 10 percent, less than 1 percent, or less than 0.1 percent) of the surface capture sites (mTet) are functionalized, leaving a large number of sites available for further reaction. The bead-oligonucleotide conjugate is then iteratively barcoded through split-pool combinatorial ligation to produce a pool of uniquely barcoded beads. A split-pool combinatorial indexing assay is implemented using these beads wherein during the first level of indexing, a reverse transcription (RT) primer bearing a pendant functionality containing the same cognate iEDDA reactive moiety (e.g. TCO) is hybridized to cell RNA. The cells are loaded in microwells, followed by the prepared barcoded beads, which bear both the barcoded oligonucleotide and the large excess of iEDDA partners (mTet). Upon cell lysis, the TCO-RT primer-cell RNA complex is captured using the rapid kinetics of the iEDDA reaction without a strict need for immediate hybridization based capture. This captured tethered RT-primer cell RNA complex can then be further elaborated as described previously.

### Example 5

### Use of a Semi-Permeable LMP Agarose Gel Overlay to Fully Compartmentalize a Cell in a Barcoded Well (or with Optional Barcode Bead)

After loading cells (an optionally barcoded beads) into microwells, a 37\hat{ }C molten pre-dissolved 2% LMP agarose in PBS gel overlay is pipetted onto a 4\hat{ } cooled microwell array and allowed to quickly gel (Wood et al, 2010; Pan et al, 2018). The thickness of the overlay is determined by the volume of reagent pipetted onto the microwell arrays and can be adjusted as necessary typically in the range of 50-300 μm. After gelation, a cell lysis mix is pipetted onto the gel overlay allowing the lysis reagents to diffuse into the microwells and lyse the cells. An incubation of 5-30 min. enables the released DNA-tagged analytes to anneal to barcoded capture probes immobilized on the surface of the well (or on a bead within the well). After lysis and annealing, the gel overlay is removed by incubating in hybridization solution spiked with 0.1-0.5 U/ml beta-agarase enzyme (NEB). In some applications, a salt tolerant beta agarase enzyme such as CaAgalbeta-agarase can be used (Hans et al., 2019). After removal of the overlay, the beads can be collected and processed as described above, or the microwell array can undergo process in a downstream assay.

### Example 6

### Antibody Oligonucleotide Staining Method

1. Human TruStain FcX (Fc Receptor Blocking Solution, Cat. No. 422301)
2. Cell Staining Buffer (BioLegend Cat. No. 420201)
3. 12×75 mm Falcon™ Round-Bottom Polystyrene Tubes (Fisher Scientific, Cat #14-959-1A or equivalent)
4. TotalSeq panel

### Protocol

1. Pellet cells at 600 g for 5 mins in a cooled 4 °C centrifuge.
2. resuspend the cells in 1 mL of 1% BSA with PBS in a microcentrifuge tube.
3. Perform a live cell count using (with Trypan Blue 1:1 dilution) an automated cell counter instrument.

| cell count | | | | |
|---|---|---|---|---|
| cell (ul) | 10 | cell count | cell/mL | viability |

| | | |
|---|---|---|
| Trypan blue(ul) | 10 | *2 |

4. aliquote cell 2 million per tube, Pellet cells at 600 g for 5 mins in a cooled 4 °C centrifuge. resuspend 2 million cells (human cells) in 90 μL of Cell Staining Buffer
5. Blocking cells.
   Add 10 μL of Human TruStain FcX™ Fc Blocking reagent. The final blocking volume should be 50 μ/per sample.
   The final blocking volume should be 50 μL. Incubate for 10 min at 4 °C.
   While cells are incubating in Fc Block, proceed to step 6.
6. Prepare antibody pool using titrated amounts (up to 1 μg) of each TotalSeq™. follow Biolegend “concentrated Liquid TotalSeq antibody cocktail staining procedure to prepare staining solution (2×)
7. Carefully pipette out the prepared antibody pool, avoiding the bottom of the tube, and add the TotalSeq™ antibody cocktail to the 100 μL blocked cell suspension.
8. Incubate for 30 minutes at 4 °C.
   transfer 100 μl of sample for each condition
9. Add 1 mL of Cell Staining Buffer and spin at 4 °C for 5 minutes at 600× g
10. Aspirate the 950 μL of supernatant, leaving ~50 μL of supernatant and pellet.
11. Add 1 mL of 1.6% PFA (prepare by diluting 16% PFA with PBS) and resuspend the cell pellet well by pipetting up and down or vortex the tube.
12. Allow the stained cells to be fixed at room temperature for 10 mins
13. Quench the fixation by adding 100 μL of 1M Tris pH 7.5 and vortex the tube immediately. Allow the tube to incubate for 5 mins at room temperature
14. Pellet the cells at 600 g for 5 mins in a cooled 4 °C centrifuge.
15. Aspirate the 950 μL of supernatant, leaving ~50 μL of supernatant and pellet.
16. Repcat Steps (13) to (15) for an additional two times. Total 3 time washes
17. Top up the stained tube to 1 mL with HSME Buffer and disperse the cell pellet with vortex or gently pipetting up and down.
18. Perform a live cell count using an automated cell counter instrument, if necessary. Example single cell protein profiling assay protocol

### Reagents:

1. SME: 1×PBS, 0.5% BSA, 0.02% Sodium Azide, 5 mM EDTA
2. HSM: SME+0.5M NaCl
3. Washing buffer: 0.1×TE+0.1% Tween20+100 mM NaCl
4. Lysis buffer: 0.1 M Tris-HCl pH 7.5, 0.5 M NaCl, 1% SDS, 10 mM EDTA
5. 10 uM Splint duplex oligo-24 plex (5 ul total, in 1-time use plate)
6. Bead collection buffer
7. Barcoded bead pool (Scale Biosciences, Inc, Modular barcode beads for microfluidic single cell genomics Cyrille L. Delley & Adam R. Abate Scientific Reports volume 11, 10857 (2021)
8. PCR primer mix

### Preparation:

1. Add 150 ul PBS+BSA into the well of plate, incubate at room temp. 1 hour; Remove buffer just before using.
2. Wash beads by washing buffer 3×
1 Add 30 ul stained cells ~10K (in HSME buffer) into each well of splint oligo plate
2 Incubate at 37 C 1 hour
3 Spin down 500 g 5 min, remove 25 ul supernatant
4 Add 100 ul HSME, spin down 500 g 5 min, remove 100 ul supernatant
5 Repeat step4 3 times
6 Pool and count the cells
7 Adjust cell concentration to about 30K/100 μL, using HSME
8 Load 100 ul cells into each well of the microwell plate
9 Wait for 5 mins, spin down 500 g 3 min
10 Gently remove supernatant from each well
11 Load about 25K beads in 150 ul Washing buffer to each well
12 Wait for at least 5 mins
13 Spin down beads at 100 g 2 mins
14 Gently remove supernatant and remaining beads from each well
15 Gently wash each well by washing buffer, then remove supernatant and remaining beads
16 repeat step 15 2×
17 Load Lysis buffer 150 ul
18 Incubate at 50c 15 min, then 37c 15 min
19 Remove lysis buffer
20 Load 100 ul beads collection buffer to each well
21 Spin 100 g 2 mins
22 Add 500 ul Washing buffer in a 1.5 mL eppendorf tube, put it on a magnetic stand
23 Pipetting 100 μL collection buffer with beads from the well to the tube from Step22
24 Wait 30 s, load 100 ul buffer from tube back to the well, without disturbing the beads on the tube wall
25 Pipetting 100 μL collection buffer with beads from the well to the tube
26 Repeat step 24-25 twice, make sure most of beads are collected
27 Remove all the supernatant
28 Add 100 ul Washing buffer, move the beads to a PCR tube on the magnetic stand
29 Wash beads twice by washing buffer
30 Remove all the supernatant
31 Add 25 μL H2O, 3 μL T4 ligase buffer, 0.5 ul T4 ligase
32 Incubate at room temp. 30 mins
33 Remove the supernatant
34 Add 25 ul H2O, 3 ul Exol buffer, 0.5 ul ExoI
35 Incubate at room temp. 30 mins
36 Remove the supernatant, washing beads 3× by washing buffer
37 Add:
   23 μL H2O
   25 μL 2× Luna PCR mix
   2uIL PCR primer mix
38 PCR

| 1 | 60 c | 5 | min |
|---|---|---|---|
| 2 | 95 c | 1 | min |
| 3 | 95 c | 20 | s |
| 4 | 60 c | 30x | |
| 5 | Go to 3 | 16-18x | |
| 6 | 60 c | 1 | min |

| | |
|---|---|
| 7 | 10 c |

for 10K cell, 16×PCR
36 SPRI clean for final library (1:1 spri to PCR mix)

### Example 7

### Single Cell 3′ Whole Transcriptome RNA Profiling Assay Protocol

### Pre-Barcoded Wells are Made as Described Above

Note: Cells are fixed with Scale Biosciences's cell fixation kit. Cells resuspended in PBST and stored at −80 C. Cell conc in 200 ul
1. Microwell plate preparation
   Add 300 μL wetting buffer
   Spin plate at 500 g, 3 min
   Incubate plate at room temperature for 1 h.
   With wetting buffer in the wells, store the plate at 4 C.
4. Barcoded microwell plate blocking
   Prepare fresh RNA blocking bufferRemove wetting buffer
Wash wells with RNA blocking buffer
Block microwell plate with RNA blocking buffer at RT, 30 min
Wash with PBST
5. Anneal splint/oligodT duplex on cells
   Set up the annealing reaction according to table below.

| Component | Stock | Final Vol(uL) | 1 | |
|---|---|---|---|---|
| Splint/OligodT duplex | 4 | 0.8 | 2 | 2.0 |
| Cells in PBST | 4 | 4.0 | | |
| Cell buffer | 4 | 4.0 | | |
| Total vol | 10 | 10.0 | | |

Incubate at 37 C 30 min with rotation.
Add cell buffer to the annealing reaction.
   Spin down at 500 g, 3 min
   Remove supernatant
   Repeat wash.
   Count cells.
   Spin down at 500 g, 3 min
   Remove supernatant
   Add cell buffer to dilute cells to 200 cells per uL
6. Cell loading
   Dilute cells hybridized with splint/OligodT duplex in cell wash buffer (200 cells per uL)
   Load cell on pre-barcoded microwell plate.
   Let cells settle at RT for 5 min
   Spin plate at 500 g, 3 min
   Remove supernatant from plate wells.
7. Microwell sealing (semi-permeable membrane overlay or bead option)
Load semi-permeable beads (not barcoded) in microwells or overlay semi-permeable membrane
Remove excess beads
8. Lysis and capture
   Prepare lysis/hyb buffer
   Add lysis buffer onto plate
   Lysis/hybridization at 37 C, 10 min.
9. Microwell washing
   Remove lysis buffer supernatant from plate
   Add wash buffer
   Spin at 300 g, 2 min.
   Remove supernatant.
   Add ligation buffer
10. Ligation
   Incubate ligation reaction at RT for 1 h
   Remove supernatant
   Wash microwells with wash buffer
   Remove wash buffer
   Add RT Buffer
11. RT and TSO reaction w/TSO
   Add reverse transcription (RT) mastermix and perform reverse transcription and template-switching
12. PCR to amply cDNA
Remove RT mastermix
Add PCR mastermix
PCR cycling
Purify PCR product by adding SPRI beads
After SPRI, elute DNA in LTE
Run sample on tapestation to check PCR product quality
Qubit to measure the concentration
Prepare final library using Fragmentation Mix
Add Fragmentation Mix (NEBNext® Ultra™ II FS DNA Module)
Follow NEB supplier protocol.
14. PCR amplify final library
Add fragmented cDNA to PCR master mix
Amplify
Purify final library
15. Sequence RNA library

### Example 8

### Single Cell 3′ Whole Transcriptome RNA Profiling Assay Protocol

Note: Cells are fixed with Scale Biosciences's cell fixation kit. Cells resuspended in PBST and stored at −80 C. Cell conc in 200 ul
1. Microwell plate preparation
   Add wetting buffer
   Spin plate at 500 g, 3 min
   Incubate plate at room temperature for 1 h.
   With wetting buffer in the wells, store the plate at 4 C.
4. Microwell plate blocking
   Prepare fresh RNA blocking buffer
Remove wetting buffer
Wash wells with RNA blocking buffer
Block microwell plate with RNA blocking buffer at RT, 30 min
Wash with PBST
5. Anneal splint/oligodT duplex on cells
   Set up the annealing reaction according to table below.

| Component | Stock | Final Vol(uL) | 1 | |
|---|---|---|---|---|
| Splint/OligodT duplex | 4 | 0.8 | 2 | 2.0 |
| Cells in PBST | 4 | 4.0 | | |
| Cell buffer | 4 | 4.0 | | |
| Total vol | 10 | 10.0 | | |

Incubate at 37 C 30 min with rotation.
Add cell buffer to the annealing reaction.
   Spin down at 500 g, 3 min
   Remove supernatant
   Repeat wash
   Count cells.
   Spin down at 500 g, 3 min
   Remove supernatant
   Add cell buffer to dilute cells to 200 cells per uL
   Cells are ready to load on microwells
6. Cell loading
   Dilute cells hybridized with splint/OligodT duplex in cell wash buffer (200 cells per uL)
   Load on microwell plate.
   Let cells settle at RT for 5 min
   Spin plate at 500 g, 3 min
   Remove supernatant from plate wells.
7. Bead loading
   Remove supernatant of bead stock solution on a magnetic stand
   Wash beads with bead wash buffer
   Resuspend beads in bead wash buffer
   Load on beads in microwell plate
   Let beads settle at RT for 5 min
   Spin plate at 200 g, 2 min.
   Remove excess beads
   Wash 2× with bead wash buffer. Make sure to wash out all excess beads.
8. Lysis and capture
   Prepare lysis/hyb buffer
   Add lysis buffer onto plate
   Lysis/hybridization at 37 C, 10 min.
9. Beads collection
   Remove lysis buffer supernatant from plate
   Add buffer
   Spin at 300 g, 2 min.
   Remove buffer with beads and add in the respective 1.5 mL tube
   Separate beads from the supernatant on the magnetic stand.
   Wash beads three times
   Resuspend beads in buffer
10. Ligation
   Incubate ligation reaction at RT for 1 h
   Magnetize bead solution
   Remove supernatant
   Wash beads with bead wash buffer
11. RT and TSO reaction w/TSO
Remove bead wash buffer
Add reverse transcription (RT) master mix and perform reverse transcription and T12. PCR to amply cDNA
Remove supernatant from beads on a magnetic stand
Remove RT master mix
Add PCR master mix
PCR cycling
Purify PCR product by adding SPRI beads
After SPRI, elute DNA in LTE
Run sample on tapestation to check PCR product quality
Qubit to measure the concentration
13. Prepare Tagmentation Mix without sample.
Add 5 μL Tagmentation Mix to each sample (sample is in 6 μL)
Shake at 2000 rpm 30 sec, pulse on microcentrifuge
Incubate at 5 min 55 C
Add tagmentation stop buffer
14. PCR amplify final library
Add tagmented cDNA to PCR mastermix
Amplify
Purify final library
15. Sequence RNA library

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

That which is claimed is:

1. An assembly comprising:
(a) a macrowell plate that comprises a single open-bottomed well; and
(b) a microwell plate comprising wells that have entrances that are in the range of 30-100 microns in diameter;
wherein:
the bottom of the macrowell plate is affixed to the top of the microwell plate;
at least 1,000 microwells of the microwell plate are exposed at the bottom of the single open-bottomed well of the macroplate; and
the wells of the microwell plate are dimensioned to allow a single bead of a defined diameter into a well but comprise a constriction that prevents the bead from coming into contact with the bottom of the well.

2. The assembly of claim 1, wherein the top of the microwell plate but not the walls of the wells of the microwell plate are hydrophobic.

3. The assembly of claim 1 or 2, wherein the walls of the wells of the microwell plate but not the top of the microwell plate are hydrophilic.

4. The assembly of any prior claim, wherein 0.5% to 95% of the wells of the microplate are exposed at the bottom of the single open-bottomed well of the macrowell plate.

5. The assembly of any prior claim, wherein the wells of the microwell plate are cone-shaped or have a step, ridge, baffle or change in wall angle that prevents the beads from coming into contact with the bottom of the wells.

6. The assembly of any prior claim, wherein the microplate has at least 1M wells.

7. A kit comprising
i. an assembly of any of claims 1-6, and
ii. beads of a defined diameter,
wherein the wells of the microwell plate are dimensioned to allow a single bead into the well but comprise a constriction that prevents the bead from coming into contact with the bottom of the well.

8. The kit of claim 7, wherein the beads are each uniquely barcoded by an oligonucleotide that is tethered thereto.

9. The kit of claim 7 or 8, wherein the beads are in the range of 20-100 microns in diameter.

10. The kit of any of claims 7-9, wherein the beads are porous and/or contain a channel.

11. The kit of any of claims 7-10, wherein the kit further comprises:
a set of at least 10 oligonucleotides that have different sequence barcodes.

12. The kit of any of claims 7-11, wherein the kit further comprises:
a container of overlay oil.

13. The kit of any of claims 7-12, wherein the kit further comprises:
a cell lysis buffer and/or a collection buffer.

14. A method for barcoding nucleic acids that are associated with biological particles, comprising:
(a) obtaining a set of compartmentalized samples that each comprise multiple biological particles, wherein the biological particles comprise nucleic acids that are associated with a first index sequence that is different in each of the compartments;
(b) combining the compartmentalized samples into a pool, thereby generating a pool of biological particles;
(c) partitioning the pool of biological particles into the wells of a microwell plate, wherein each well of the microwell plate that receives a biological particle receives an average of one or more biological particles;
(d) compartmentalizing the biological particles in the wells by distributing a population of beads across the wells, wherein:
(i) each well receives, on average, a single bead;
(ii) the beads enter the wells but do not come into contact with the bottom of the wells, thereby encapsulating the particles in compartments; and
(iii) the beads are uniquely barcoded by oligonucleotides that are tethered to the beads and comprise a second index sequence that is different for each of the beads;
(e) lysing the biological particles while they are in the compartments to release the nucleic acids that comprise the first index sequences into solution;
(f) incubating the microwell plate under conditions sufficient for the released nucleic acids to form complexes with the oligonucleotides that are tethered to the beads in the compartments, to produce beads that have complexes comprising a first index sequence and a second index sequence;
(g) pooling the beads; and
(h) performing an en masse molecular reaction on the beads to produce reaction products that contain a first index sequence or complement thereof and a second index sequence or complement thereof.

15. The method of claim 14, further comprising:
(i) sequencing the reaction products, or an amplification product thereof, to produce sequence reads.

16. The method of claim 15, further comprising:
(j) grouping together sequence reads that have the same first index sequence or complement thereof and the same second index sequence or complement thereof.

17. The method of claim 16, wherein the combination of i. the first index sequence or complement thereof and ii. the second index sequence or complement thereof identify a single biological particle from a particular sample.

18. The method of any of claims 14-17, wherein the molecular reaction is done by ligation, primer extension and/or gap-fill ligation.

19. The method of any of claims 14-18, wherein the biological particles are cells or nuclei isolated from cells.

20. The method of claim 19, wherein combination of the first index sequence or complement thereof and the second index sequence or complement thereof identify a single cell or nucleus from a particular sample.

21. The method of any of claims 14-20, wherein the beads are porous or have channels and step (e) comprises flooding the microwell plate with a lysis buffer that diffuses through the beads into the compartments.

22. A method to generate a pre-barcoded microwell array comprising:
(a) obtaining:
an activated microwell array comprising wells that have diameters that are in the range of 30-100 microns in diameter;
barcoded beads with a diameter matched to the diameter of the microwell array such that only a single bead can load in any given microwell;
a population of activated and reversibly attached oligonucleotide barcodes on the barcoded beads.
(b) assembling barcoded beads (b) into the microwell array of (a), wherein a majority of the wells receives a single bead per well;
(c) releasing the activated oligonucleotide barcodes from the beads in the wells;
(d) coupling of the activated oligonucleotide barcode to the activated microwell surface;
(e) removing the beads from the microwells.

23. A method to combinatorial index a single cell/nuclei by the following steps:
(a) partitioning biological particles into the microwells of a pre-barcoded microwell array with an average of 1 or more particle per microwell;
(b) lysing contents of biological particles;
(c) ligating pre-indexed sample analytes onto pre-barcoded oligonucleotide tethered to microwells;
(d) Optionally performing primer extension reaction to create a copy of the complete ligated bi-indexed complexes;
(e) eluting copy of bi-indexed complexes from microwells and pool;
(f) amplify and NGS sequence resultant libraries.
